(19) United States
(12) Patent Application Publication
(10) Pub. No.: US 2013/0045457 A1
(43) Pub. Date: Feb. 21, 2013

(54) Lighting Device for Teeth Whitening

(71) Applicants: Ramswamy Rangraj Chetiar, Monroe, NY (US); Viswaraj Ramswamy Chettiar, Monroe, NY (US); Bashker Rangaraj Chetiyaar, Monroe, NY (US)

(72) Inventors: Ramswamy Rangraj Chetiar, Monroe, NY (US); Viswaraj Ramswamy Chettiar, Monroe, NY (US); Bashker Rangaraj Chetiyaar, Monroe, NY (US)

(73) Assignee: MAGICSMILE USA, INC., Monroe, NY (US)

(21) Appl. No.: 13/294,087

(22) Filed: Nov. 10, 2011

Related U.S. Application Data
(60) Provisional application No. 61/490,123, filed on May 26, 2011.

(51) Int. Cl. A61N 5/06

(52) U.S. Cl. 433/29

(57) ABSTRACT

A safe, quick, efficient and easy to use lighting device for teeth whitening is disclosed. The lighting device has a plurality of LEDs that emit lights of a wavelength ranging between 490 nm to 505 nm. The color of the light may be blue, blue green, green, aqua or cyan. A mouth tray may be prefilled with a predetermined quantity of whitening gel and is attached firmly to the lighting device and placed inside the mouth of the patient. The lighting device switches ON only when new mouth tray is attached to it and it switches OFF after using it for 30 minutes. Teeth are cleaned from the front and inner side since the gel spreads throughout the jaw of the patient. The same procedure may be repeated after 24 hours since the procedure doesn't cause tooth sensitivity or tissue burning and is safe even if used frequently and gives instant results.

### CROSS-REFERENCE

The present invention claims priority to Provisional Application Ser. No. 61/490,123 filed May 26, 2011, entitled “Lighting Device for Teeth Whitening”, which is hereby incorporated by reference.

### TECHNICAL FIELD OF THE INVENTION

The invention relates to a device for teeth whitening. More particularly, the invention relates to an LED (Light Emitting Diode) based lighting device for whitening of teeth efficiently and quickly without expert supervision.

### BACKGROUND OF THE INVENTION

The most common causes of discoloration of teeth include aging, consumption of staining substances. As a person ages, the adult teeth often become darker due to changes in the mineral structure of the tooth as the enamel becomes less porous. Teeth can also become stained by bacterial pigments, foodstuffs and tobacco. Certain antibiotic medications (like tetracycline) can also cause teeth stains or a reduction in the brilliance of the enamel. The trend of tooth whitening has gained popularity these days and as a result plenty of devices, applicators or compositions have flooded the markets. Bleaching of teeth is a popular and widely used procedure which is useful in lightening discolorations which have accumulated on the tooth surface over a period of time. Various devices and methods available in the market include various bulky and high intensity devices. Power or light-accelerated bleaching, sometimes colloquially referred to as laser bleaching, uses light energy to accelerate the process of bleaching for teeth whitening. Such bleaching devices make use of a light source and a flexible dental tray that is removably attached to it. The source of energy for the purpose is generally a high energy device to excite the peroxide molecules without overheating the pulp of the tooth. Lights are typically within the blue light spectrum as this has been found to contain the most effective wavelengths for initiating the hydrogen peroxide reaction. A tooth whitening agent is placed on the dental tray and the dental tray is pressed onto the user's teeth. The light source fixed to the dental tray is projected towards it from outside. The heat produced by the light source causes chemical reaction with the whitening agent upon activating the device. The whitening agent is permitted to bathe or coat the teeth and the chemical reaction enhances and/or expedites the tooth whitening effect of the application.

While such devices are less expansive and compact than professional devices and also are quite effective in bleaching the teeth to a certain extent but suffer from some serious side effects that include: chemical burns from gel bleaching (if a high-concentration oxidizing agent contacts unprotected tissues of gums, which may bleach or discolor mucous membranes), increased sensitiveness of teeth, over bleaching known as Hyperodonto-oxidation, pain if the patient has “sensitive teeth” caused by open dentinal tubules and increased risk of tongue cancer. Moreover, the high intensity light sources generally burns the soft gum tissues around the teeth and the areas surrounding the mouth such as lips as well since there is no mechanics available that can prevent the exposure of high wavelength lights to these delicate portions.

Apart from above mentioned drawbacks, these devices suffer from one more drawback that these devices are manufactured to clean only the front portion of the teeth and often neglect the inner side of the teeth during the procedure. Internal staining of dentine can discolor the teeth from inside out. Generally a separate and extensive procedure is needed to clean the interior side of the teeth and all the device and methods available in the market are unable to whiten teeth from the interior side of the teeth in the same sitting as is done for front teeth cleaning.

Hence what is needed is a device and a method for whitening teeth which is portable, lightweight, efficient and provides a safe method to whiten the teeth by overcoming all the side effects of the prior art.

### BRIEF DESCRIPTION OF THE DRAWINGS

FIG. 1 illustrates an isometric view of the lighting device and an exemplary mouth tray used with the lighting device in accordance with one embodiment of the invention.

FIG. 2 illustrates an isometric view of the lighting device in accordance with one embodiment of the invention.

FIG. 3 illustrates a top view of the lighting device in accordance with one embodiment of the invention.

FIG. 4 illustrates a bottom view of the lighting device in accordance with one embodiment of the invention.

FIG. 5 illustrates a side view of the lighting device in accordance with one embodiment of the invention.

FIG. 6 illustrates a front view of the lighting device in accordance with one embodiment of the invention.

FIG. 7 illustrates rear view of the lighting device in accordance with one embodiment of the invention.

FIG. 8 illustrates a top view of the inside components of the lighting device in accordance with one embodiment of the invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

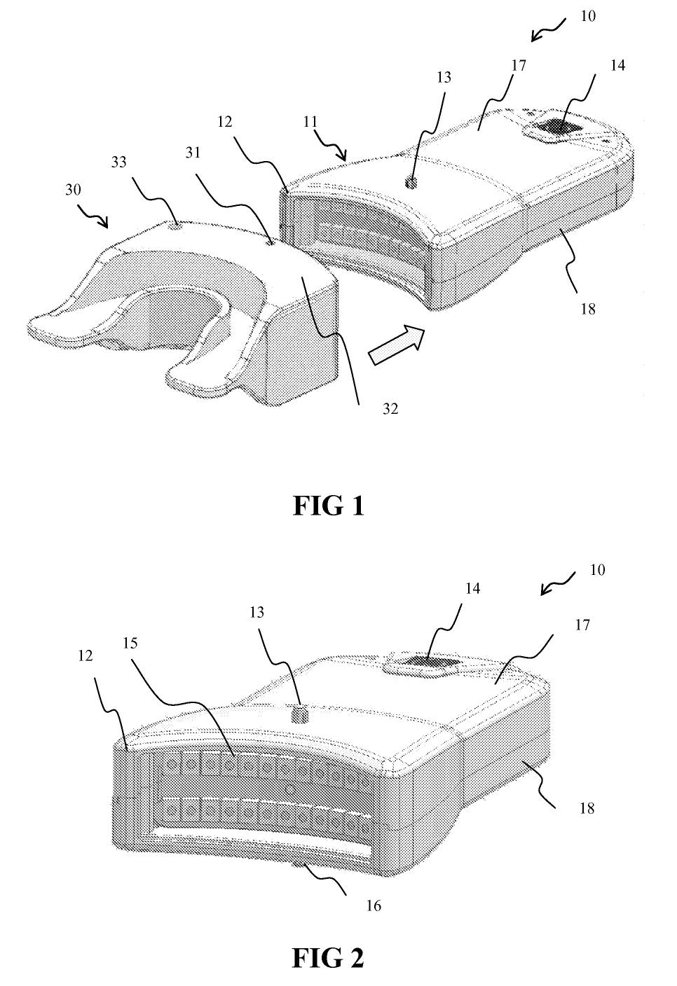

FIG. 1 illustrates an isometric view of the lighting device and an exemplary mouth tray used with the lighting device in accordance with one embodiment of the invention. The lighting device 10 has a plurality of LEDs on the front portion 12 that emit lights of a particular wavelength. The lighting device 10 is used in conjunction with a mouth tray 30 as illustrated in the Figure. Already existing mouth trays can also be used with the lighting device 10 with slight modifications to the structure. The mouth tray 30 is detachably attached to the lighting device 10 by the locking mechanism provided on the front portion 12 of the housing 11 of the lighting device 10. A predetermined quantity of whitening gel is placed on the cavities of the mouth tray 30 or the mouth tray 30 may be packaged by the distributer in such a way that a predetermined quantity of the gel is already placed on each of the mouth trays 30 and is sealed and sold in the market in ready to use packets. The consistency of the gel is such that it doesn't drip or flow after being placed on the mouth tray 30. Once the mouth tray 30 fits within the mouth of the patient it covers all the upper and lower teeth. The lighting device 10 is then switched ON by pressing the ON/OFF switch 14 provided on the upper housing 17 of the lighting device 10. Since the illuminating portion i.e. the upper portion 12 of the lighting device 10 faces the mouth tray 30, the light reacts with the gel. The gel contains photo initiators that accelerate the process of disintegration of the gel into water and oxygen. At the same time, the gel starts melting upon disintegrating and spreads across the front and rear side of both, the upper and lower teeth line. Since teeth have microscopic pores on their surfaces and when stain causing foods and drinks get into these pores, teeth tend to darken over the years. The oxygen released by the disintegration of the gel enters into the teeth pores and converts the colored compounds inside the pores to uncolored compounds. In other words, the oxygen bleaches the colored compounds inside the teeth pores. In most preferred embodiment, the top portion of the upper housing 17 has a first elevated portion 13 for engaging the mouth tray 30 to the lighting device 10. The bottom housing 18 of the lighting device 10 also has a second elevated portion 16 just opposite to the first elevated portion 13 on the other side.

In preferred embodiments, the mouth tray 30 to be used with the device must be such that there should be provided an extended portion 32 towards the lower end and a slot 31 of suitable diameter may be provided on this extended portion 32 to accept the first elevated portion 13 and the second elevated portion 16 on the lighting device 10. The mouth tray 30 is attached to the lighting device 10 by bringing them towards each other and inserting the elevated portion 13 inside the slot 31 on the mouth tray 30. The mouth tray 30 attaches firmly to the lighting device 10 due to an additional support provided by the extended portion 32 of the mouth tray 30 and prevent the lighting device 10 from slipping or misalignment during the procedure. Since the mouth tray 30 secures firmly to the lighting device 10, the patient doesn't need to support the lighting device 10 with his hands. The extended portion 32 also prevents exposure of the light from the LEDs to the areas surrounding patient's mouth including accidental exposure to eyes as well. In another embodiment, the already available mouth trays can also be attached to the lighting device 10 by using other suitable mechanisms. The whole procedure takes about 30 minutes without causing tooth sensitivity and without exposing the delicate portions of the mouth and gums to the light released from the lighting device 10.

FIG. 2 illustrates an isometric view of the lighting device in accordance with one embodiment of the invention. The lighting device 10 comprises the front portion 12 having a plurality of Light Emitting Diodes 15 and a housing to enclose the battery and various electronic components. The front portion 12 is slightly semicircular in shape in order fit exactly to the curve of the mouth tray 30 that further fits to the human jaw. The lighting device 10 has two Printed Circuit Board Assemblies. One is the LED (Light Emitting Diode) Printed Circuit Board Assembly (LED PCBA 19a) and the other is called the Driver Printed Circuit Board Assembly (Driver PCBA 19b) (The driver PCBA 19b is explained with respect to FIG. 8). The front portion 12 of the lighting device 10 includes a plurality of light emitting diodes (LEDs) 15 arranged in a row on a printed circuit board (PCB) to emit light on the teeth. The closely spaced light emitting diodes 15 are connected in serial or in parallel and are arranged in two parallel rows along the longitudinal axis of the front portion 12 so as to cover both, the upper teeth line and the lower tooth line of the human jaw. The preferred angular range of the light emitted from the light emitting diodes 15, according to the present invention, is approximately 120 degree in the direction in which the light from the light emitting diodes 15 is emitted. The color of the light emitted from the LEDs 15 is any of these blue, blue green, green, aqua or cyan. In most preferred embodiment, the light emitted from the LEDs are selected such that the wavelength remains in the range of 490 nm to 505 nm which is most suited for reacting with the whitening gel. The intensity of the LEDs 15 is preferably between a range of 1500 mcd to 2200 mcd. The intensity of the LEDs 15 is controlled by the illuminated/non illuminated push button/tactile switch provided on the upper housing 17 of the lighting device 10. Hence when it is need to whiten a comparatively darker shade of teeth, the intensity of the light emitted from the LEDs 15 can be increased to a required value. Upon increasing the intensity, more intense light falls of the whitening gel and intensifies the reach of oxygen molecules to the deeper pores of the teeth. In illustrated embodiment, 26 LEDs are used for the purpose of illustration whereas the number of LEDs may vary depending upon various conditions. The main advantage of using LEDs is that it doesn't generate heat in spite of increasing the intensity while a sufficient amount light can be generated by consuming a small amount of power. Thus teeth and gums are prevented from undesirable damage. The lighting device 10 runs on a 3.7 volts, Lithium-Ion or Lithium Polymer Battery of capacity ranging from 1200 mah to 1500 mah only. Since the battery required to power the LEDs is quite a low powered and is comparatively of much lower weight as compared to the available devices that provide a separate unit for battery which are in turn connected to the lighting instrument separately through a wire and the like. The lighting device 10 of the present invention provides a compact, lightweight and easy to use lighting device for teeth whitening. The light weight feature also removes the need of supporting the device with hands during the use of the device. The lighting device 10 can be easily fixed to the mouth tray and can be used without any external support. The patient is free to move in any direction during the procedure.

The dental whitening is achieved by applying simultaneously the light from the lighting device 10, disintegrating the whitening gel, opening the pores of the teeth and discoloring the colored composites inside the pores. The lighting device 10 provides a quick and efficient method of whitening teeth while completely removing the need of multiple sitting and pre-operative preparations.

The housing 11 of the lighting device 10 is made of ABS/PC or PC+ABS plastic material which is completely water and dust proof type so as to protect the LEDs from the saliva falling from the mouth of the patient during the procedure. The water proofing of the device can be accomplished either by ultrasonic welding process of top and bottom plastics covers or with the use of water proof gaskets sandwiched between the top and bottom portion of the outer covers.

The patient is free to make any movements during the procedure and has sufficient mobility to move in any direction, read or write. After the procedure is over, the mouth tray can be disposed of and a new mouth tray prefilled with the whitening gel can be used by another person. Since the mouth tray is prefilled with a measured quantity of the whitening gel, the chances of using over filled or under filled mouth tray are diminished. In traditional methods, refills are provided to the users wherein they can refill the mouth tray after being used once. In case a user performs the procedure at home without an expert supervision, he/she might end up using the wrong quantity of the whitening gel which may have adverse effects.

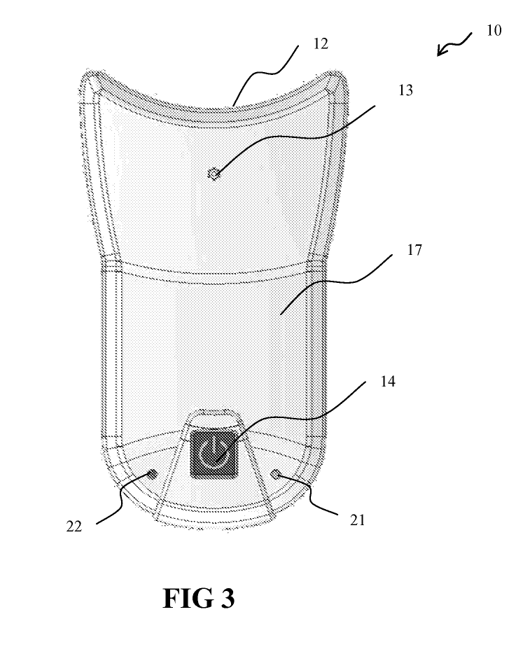

FIG. 3 illustrates a top view of the lighting device in accordance with one embodiment of the invention. As seen from the figure, the front portion 12 of the lighting device 10 has a slightly semicircular shape so as to adapt properly to the parabolic of a standard mouth tray. In one embodiment, the first elevated portion 13 on the upper housing 17 of the lighting device 10 may be provided to attach the mouth tray to the lighting device 10. The lower portion of the housing of the lighting device 10 is preferably molded in an ergonomic curved shape for comfortable usage. A green color LED 22 of approximately 3 mm size may be used to indicate the Battery Charging status of the lighting device 10. Another red color LED 21 of approximately 3 mm size may be used to indicate the low battery level.

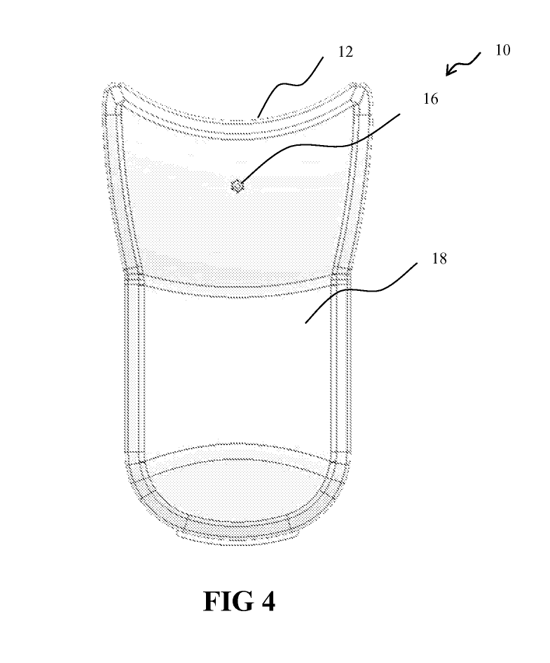

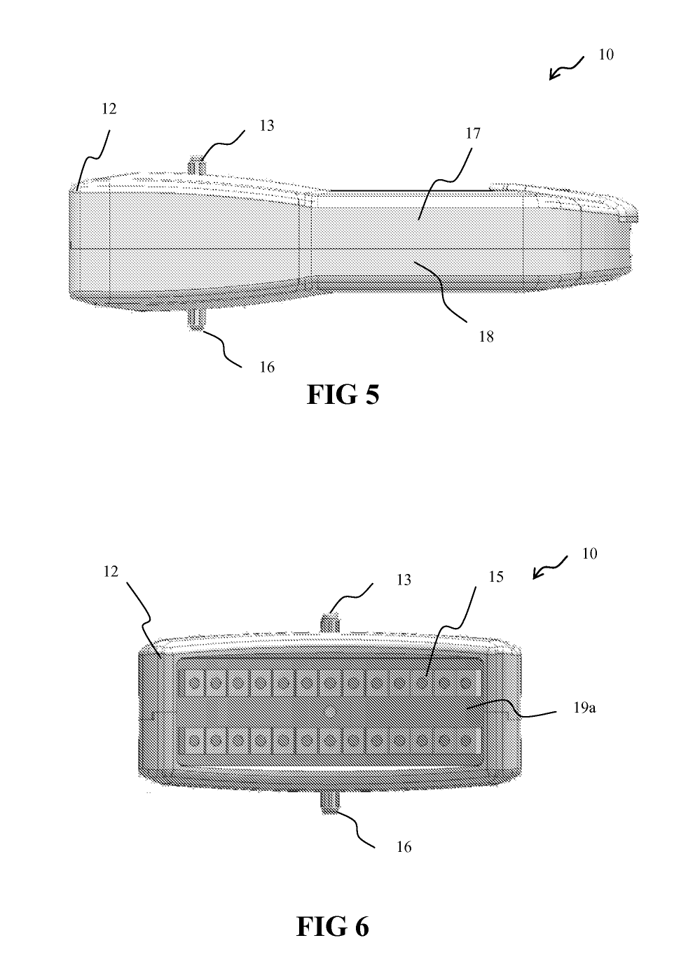

FIG. 4 illustrates a bottom view of the lighting device in accordance with one embodiment of the invention; and FIG. 5 illustrates a side view of the lighting device in accordance with one embodiment of the invention. As, shown in FIG. 4, the bottom housing 18 of the lighting device 10 has a second elevated portion 16 which is just opposite in direction to the first elevated portion 13 on the other side (as shown in FIG. 5). The slots 31 (FIG. 1) on the extended portion 32 (FIG. 1) of the mouth tray 30 (FIG. 1) tightly engage the both the first elevated portion 13 and the second elevated portion 16 of the lighting device 10 in order to keep it in place during the procedure and prevent undesirable exposure of the light to vulnerable areas of face and mouth.

FIG. 6 illustrates a front view of the lighting device in accordance with one embodiment of the invention. For the purpose of illustration, only 13 LEDs are shown on each row whereas the number of LEDs may vary depending upon various conditions. The LED PCBA 19a is made up of 0.2 to 0.3 mm flexible or rigid epoxy PCB mounted on the thin ABS Plastic sheet with thickness of approximately 1 mm to 1.6 mm. The LED PCBA 19a is connected to the Driver PCBA 19b (FIG. 8) by thin copper PVC Flexible wires.

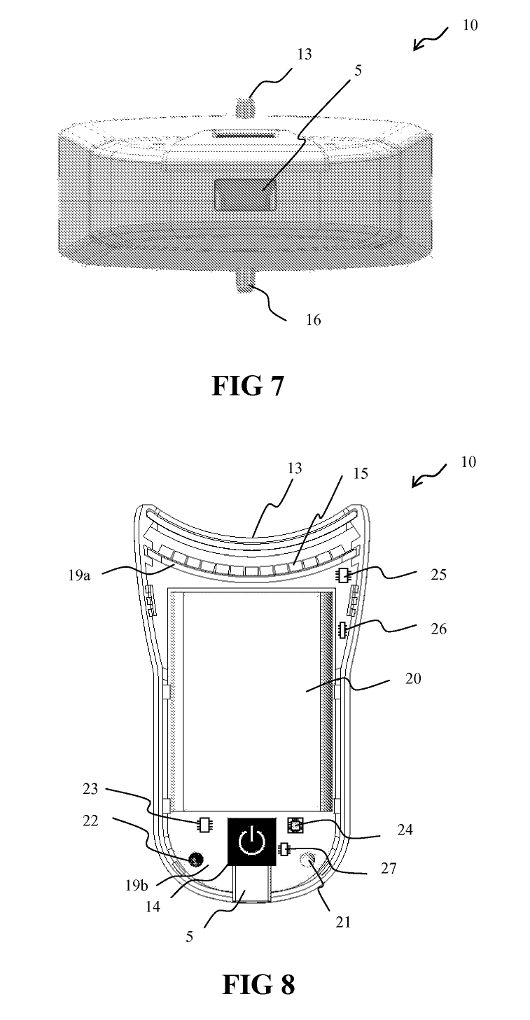

FIG. 7 illustrates rear view of the lighting device in accordance with one embodiment of the invention. The rear portion of the lighting device 10 may include at least one port 5 for recharging the battery using a suitable AC adapter or a USB port of the desktop computer, laptop/Notebook or any other external device. Examples of the port include, but not limited to a mini Universal Serial Bus (USB) port, an Ethernet port and so forth. It is well understood for a person skilled in the art that various other mean for wirelessly recharging can also be used.

FIG. 8 illustrates a top view of the inside components of the lighting device in accordance with one embodiment of the invention. The electronics involved in the lighting device 10 comprises mainly of a driver PCBA 19b which is basically a PCB (Printed Circuit Board) having 1 to 1.6 mm thickness, glass epoxy, FR4 type, and is green masked with white silk screen printed of the size that can be fitted within the dimensions of the device. An approximately 3.7 volts Lithium-Ion or Lithium Polymer Battery 20 of capacity preferably ranging from 1200 mah to 1500 mah is used. The port 5 is preferably a mini Universal Serial Bus (USB) port, or an Ethernet port which is used to recharge the rechargeable battery. A green color LED 22 of approximately 3 mm size may be used to indicate the Battery Charging status of the lighting device 10. Another red color LED 21 of approximately 3 mm size may be used to indicate the low battery level. The ON/OFF switch 14 may preferably be an illuminated or non-illuminated tactile/push button switch to turn ON or turn OFF the lights of the lighting device 10. In order to boost the output voltage of the battery 20, a boost converter IC (Integrated circuit) may be used that ensures a constant drive voltage to the LEDs even if the battery voltage drops to a lower level during the time LEDs are ON or while discharging. A boost converter (step-up converter) 23 is a standard power converter with an output DC voltage greater than its input DC voltage. An LED driver transistor 25 may be disposed to drive the LEDs. The micro controller 26 used in the lighting device 10 may be an 8 bit or a 16 bit or any equivalent. A USB Battery Charger IC (Integrated Circuit) 24 may be used that will correctly and safely charge the Lithium Polymer battery 20 with power from the port 5. Maximum current is limited to 300 mA using an external wall supply (3.7V to 7V). The Battery Charger IC 24 requires almost no external components and will automatically select between a DC source and USB source if both are present. Intensity of the light may adjustable to at least three levels. The entire unit is controlled by a Nano watt technology microcontroller/microprocessor. The light produced by the LEDs is capable of producing constant luminous output irrespective of the battery voltage down to 2.4 volts.

The lighting device 10 is also fitted with a Radio Frequency Identification (RFID) reader 27 to read the identification code of the RFID tag 33 (FIG. 1) disposed on the extended portion 32 (FIG. 1) of the mouth tray 30 (FIG. 1) to be used in conjunction with the lighting device 10. A 3 mm circular RFID tag 33 (FIG. 1) is embedded on the mouth tray 30 (FIG. 1). According to an embodiment of the present invention, the mouth tray having an RFID tag therein as described in Application entitled “Mouth Tray for Teeth Whitening” by Ramswamy Rangraj Chetiar et al. may be used and is being incorporated herein by reference. The RFID tag 33 (FIG. 1) is preferably of Low frequency type (134 khz) or High Frequency type (13.56 mhz).

Each RFID tag 33 (FIG. 1) is coded with a unique identifier to distinguish it from other RFID tags. Radio-frequency identification (RFID) uses communication through the use of radio waves to exchange data between a RFID reader 27 and the RFID tag 33 (FIG. 1) attached to the mouth tray for the purpose of identification and tracking. Upon fitting a new mouth tray 30 (FIG. 1) to the front portion 12 of the lighting device 10, the Radio Frequency Identification (RFID) reader 27 communicates with the RDIF tag 33 (FIG. 1) and decodes the unique identifier associated with it. The lighting device 10 is switched ON only if it finds a mouth tray with new identifier. In case the RFID reader 27 detects a RFID tag with the identifier similar to the one previously used, the RDIF reader 27 stops the power supply to the lighting device 10. Thus unless a new mouth tray is not used, the lighting device 10 doesn't switch ON. This feature is added in the device in order to ensure that the same mouth tray is not used after being used once for a patient. Hence, ensuring that each mouth tray is used only once and is disposed of after single use. The lighting device 10 may be programmed to be switched ON only for 30 minutes once it finds a new mouth tray with new identifier. This feature ensures the over exposure of the lighting device in case the patient is using the device at home without an expert supervision.

The system described above totally eliminates the need of an expert supervision by providing a safe, efficient and easy to use lighting device for teeth whitening. In one embodiment, the device can be used with already existing mouth trays with slight modifications to the mouth tray whereas in another embodiment, the mouth tray can be embedded with RFID tags as well to ensure the same mouth tray is not used twice. This feature totally eliminates the damage caused by over application of the whitening gel since only the mouth trays prefilled with measured quantities of the whitening gel can be used with the lightening device. The lighting device cleans teeth from the front side as well as inner side since the gel spreads throughout the jaw of the patient and reaches the interior portions of the teeth. None of the existing methods or devices have the capability of cleaning teeth from the inner side of the jaw.

Moreover, there is no need of multiple sittings or pre-operative preparations before the procedure of teeth whitening and the method is quick and efficient and gives instant results. In case the patient needs another session, the same procedure may be repeated after just 24 hours since the procedure doesn't cause tooth sensitivity or tissue burning and is totally safe even if used frequently. The lighting device is quite user friendly and easy to operate. The light weight of the device also removes the need of supporting the device with hands during the procedure. The lighting device can be easily fixed to the mouth tray and can be used without any external support. The patient is free to move in any direction during the procedure. The automatic switching OFF feature of the lighting device ensures complete safety of the patient in case of accidental overexposure. Since the lighting device switches ON only after attaching the mouth tray, the exposure of bright lights to the eyes of the patient is totally eliminated since the mouth tray covers all the sides of the upper portion of lighting device. Since the lighting device is rechargeable, there is no need to carry extra batteries or other equipment and makes the device portable and easy to use.

1. A portable lighting device for teeth whitening, comprising:
a housing comprising a front portion, a rear portion, an upper housing portion and a bottom housing portion;
a plurality of lighting members on the front portion of the housing and configured to emit light of a predetermined wavelength;
a locking mechanism disposed at the upper and bottom housings, respectively, to attach a detachable mouth tray to the front portion of the lighting device; and
a power supply unit configured to supply power to the lighting device.

2. The portable lighting device of claim 1, wherein the locking mechanism comprises:
a first elevated portion formed at the upper housing portion and a second elevated portion formed at the bottom housing portion opposite a direction of the first elevated portion, for attaching and securing the mouth tray to the lighting device.

3. The portable lighting device of claim 1, wherein the power supply unit comprises a battery having a capacity ranging from approximately 1200 mah to approximately 1500 mah.

4. The portable lighting device of claim 3, wherein the battery is a lithium-ion or lithium polymer battery.

5. The portable lighting device of claim 1, further comprising:
a first printed circuit board assembly and a second printed circuit board assembly connected together wherein the plurality of lighting members are disposed on the first printed circuit board assembly and are connected in series or in parallel, and are arranged in two parallel rows along a longitudinal axis of the front portion.

6. The portable lighting device of claim 1, wherein the wavelength ranges from approximately 490 nm to approximately 505 nm.

7. The portable lighting device of claim 1, wherein the housing is formed of a waterproof plastic material.

8. The portable lighting device of claim 7, wherein the waterproof plastic material is ABS/PC material or PC+ABS plastic material.

9. The portable lighting device of claim 1, wherein the front portion of the housing is formed of a semicircular shape to adapt to the mouth tray.

10. The portable lighting device of claim 5, wherein the first printed circuit board assembly is made of a flexible or rigid epoxy and mounted on a plastic sheet.

11. The portable lighting device of claim 10, wherein the epoxy comprises a thickness ranging from approximately 0.2 to 0.3 mm and the plastic sheet comprises a thickness ranging from approximately 1 mm to approximately 1.6 mm.

12. The portable lighting device of claim 5, wherein the second printed circuit board assembly comprises:
at least one port for recharging the power supply unit via an external device;
the power supply unit;
a switching device configured to switch the lighting device between an non-illuminating state and an illuminating state and to control and intensity of the lighting members;
a power converter configured to provide a constant drive voltage to the lighting members when a power supply level decreases while the lighting device is in the illuminating state or while discharging;
a lighting member driver configured to drive the lighting members;
a microcontroller configured to control the lighting device; and
a charging unit configured to charge the power supply unit.

13. The portable lighting device of claim 12, wherein the charging unit is configured to automatically select between an internal DC source and power supplied by the external device via the at least one port.

14. The portable lighting device of claim 12, wherein the intensity ranges from approximately 1500 mcd to approximately 2200 mcd.

15. The portable lighting device of claim 12, wherein the second printed circuit board assembly further comprises:
a plurality of status light members configured to indicate a power level and charging level of the battery.

16. The portable lighting device of claim 1, further comprising a radio frequency identification (RFID) reader configured to read and decode and identifier of a RFID tag disposed on the mouth tray.

17. The portable lighting device of claim 16, wherein the lighting device switches to the illuminating state only when a mouth tray having a new identifier and attached to the lighting device, is detected by the RFID reader, and maintaining the lighting device in the non-illuminating state when a previously read RFID tag of an attached mouth tray is detected by the RFID reader.

18. The portable lighting device of claim 17, wherein the lighting device is programmable to switch to the illuminating state for a predetermined time period when a mouth tray having a new identifier is attached to the lighting device.

19. The portable lighting device of claim 1, wherein the lighting members are light emitting diodes.
